(19) United States
(12) Patent Application Publication
(10) Pub. No.: US 2010/0185271 A1
(43) Pub. Date: Jul. 22, 2010

(54) DELIVERY APPARATUS FOR A RETRACTABLE SELF EXPANDING NEUROVASCULAR STENT

(75) Inventor: Yi Zhang, San Diego, CA (US)

Correspondence Address: HIGGS, FLETCHER & MACK LLP, 401 West A Street, Suite 2600, SAN DIEGO, CA, 92101, US

(73) Assignee: Achieva Medical (Shanghai) Co., Ltd., Shanghai (CN)

(21) Appl. No.: 12/686,988

(22) Filed: Jan. 13, 2010

(30) Foreign Application Priority Data
Jan. 19, 2009 (CN) 200910045521.8

(51) Int. Cl. A61F 2/84

(52) U.S. Cl. 623/1.11

(57) ABSTRACT

The present invention relates to a delivery apparatus for delivering a self-expanding neurovascular stent that allows for smooth movement of the apparatus along a typically tortuous vascular path, ease of stent deployment, and ease of stent retractability being pushed and pulled through the delivery apparatus. The apparatus includes an outer catheter, and an inner shaft located coaxially within the outer catheter. The stent is mounted on the distal section of the inner shaft and preloaded within the outer catheter distal region. The inner shaft includes at least one stent blocking member disposed in the distal section. The self-expanding stent has proximal, middle and distal ends and is comprised of a plurality of closed cells. The self-expanding stent includes locking members which interlock with the blocking member(s) disposed on the inner shaft so as to lock the stent onto the inner shaft within the outer catheter, and to enable the stent retractable together with the inner shaft being out of and retrieved back to the outer catheter. More specially, the invention may be used in the treatment of blood vessel blockage and aneurysms which occur in the brain.

### PRIORITY DOCUMENTS

This Application claims the benefit of Chinese patent application number 200910045521.8, filed Jan. 19, 2009, which is incorporated herein by reference.

### FIELD OF THE INVENTION

The present invention relates to a neurovascular stent delivery apparatus for delivering a self-expanding neurovascular stent to a neurovascular target site, that allows for smooth movement of the apparatus along a typically tortuous vascular path, ease of stent deployment, and ease of stent retractability being pushed and pulled through the delivery apparatus.

### BACKGROUND OF THE INVENTION

It is well documented that annually in the United States, approximately 780,000 patients will experience new or recurring stroke. Stroke affects the arteries leading to and within the brain. A stroke occurs when a blood vessel that carries oxygen and nutrients to the brain is either blocked by intracranial atherosclerotic clot (ischemic stroke) or bursts (hemorrhagic stroke). When that happens, part of the brain cannot get the blood (and oxygen) it needs, so it starts to die. Of these strokes, approximately 88% are diagnosed as ischemic in nature, while the remaining 12% are attributable to hemorrhagic events.

Successful stenting for the management of coronary atherosclerotic stenosis has sparked intense interest in research and development of neurovascular stents for intracranial indications. Safety of balloon-expandable stenting is often compromised by limited flexibility of the balloon-mounted stent delivery systems, high inflation pressure required to deploy stent in fragile intracranial vessels, the risk of shearing the stent off the balloon while navigating to the target lesion, and by difficulty in accurately sizing the stent to the vessel diameter. Balloon-expandable stents have been largely replaced by self-expanding stent technology, because of self-expanding stent's ease of use, better deliverability, and lower tendency for vessel rupture and damage to the artery during deployment. (See e.g., Higashida R T, Tsai F Y, Halbach V V, et al. Transluminal angioplasty, thrombolysis, and stenting for extracranial and intracranial cerebral vascular disease. J Intervent Cardiol 1996;9:245-256; Han P P, Albuquerque F C, Ponce F A, et al. Percutaneous intracranial stent placement for aneurysms. J Neurosurg 2003;99:2330; Lylyk P, Ceratto R, Hurvitz D, Basso A. Treatment of a vertebral dissecting aneurysm with stents and coils. Neurosurgery 1998;43:385-388; Lylyk P, Cohen J E, Ceratto R, et al. Endovascular reconstruction of intracranial arteries by stent placement and combined techniques. J Neurosurg 2002;97:1306-1313; and Mericle R A, Lanzino G, Wakhloo A K, et al. Stenting and secondary coiling of intracranial internal carotid artery aneurysm. Neurosurgery 1998;43:1229-1234). Although a variety of intravascular self-expanding stents have been proposed heretofore, for example, in U.S. Pat. No. 4,665,906 issued to Jervis and U.S. Pat. No. 4,925,445 to Sakamoto et al, designing delivery systems for delivering self-expanding stent has proven difficult. Accordingly, the prior art teaches various methods and apparatuses for delivering the self-expanding stents. Examples of such delivery methods and apparatuses can be found in U.S. Pat. No. 4,580,568 issued to Gianturco; U.S. Pat. No. 7,169,170 issued to Widenhouse; and U.S. Pat. No. 7,311,726 issued to Mitelberg et al.

U.S. Pat. No. 4,580,568 discloses a delivery apparatus which uses a hollow sheath, like a catheter. The sheath is inserted into a body vessel and navigated there-through so that its distal end is adjacent the target site. A self-expanding stent is then compressed to a smaller diameter and loaded into the sheath at the sheath's proximal end. A cylindrical flat end pusher, having a diameter almost equal to the inside diameter of the sheath is inserted into the sheath behind the stent. The pusher is then used to push the stent from the proximal end of the sheath to the distal end of the sheath. Once the stent is at the distal end of the sheath, the sheath is pulled back, while the pusher remains stationary, thereby exposing the stent and allowing it to expand within the vessel. However, delivering the stent through the entire length of the catheter may cause many problems, including possible damage to a vessel or the stent during its travel. The small and tortuous neurovascular pathway makes this system even more problematic because the pathway increases the risk of damage to the vessels and/or stent. U.S. Pat. No. 7,169,170 describes a prior art pre-loaded self expanding stent delivery system wherein the stent is pre-loaded into the distal end of the outer catheter and the outer catheter and preloaded stent are simultaneously delivered through a pathway and to a target site. This patent discloses a delivery apparatus to deliver the pre-loaded self-expanding stent to a target site. Once the physician determines that the stent is sufficiently placed about the target site, the outer sheath is pulled back proximately, while the inner shaft is held in a fixed position. The stop on the inner shaft prevents the stent from sliding back with the outer sheath, so as the sheath is moved back, the stent is effectively “pushed” out of the distal end of the sheath. But, frequently these preloaded stents are misdeployed, meaning that the stent is not at the optimal position at the target site, because of either less that optimal placement of the outer catheter to the target site and/or because of movement of the apparatus during the deployment.

One important factor for controlled delivery of the stent is having a precisely controlled retraction of the retractable outer sheath. Some of the technical difficulties of the known delivery apparatus are misdeployment of the self-expanding stent if it was either too distal or too proximal to the target lesion. Accordingly, the prior art teaches various methods and apparatuses for repositioning a self-expanding stent. Examples of such methods and apparatuses can be found in U.S. Pat. No. 6,267,783 issued to Letendre et al.; U.S. Pat. No. 6,843,802 issued to Villalobos et al., U.S. Pat. No. 7,001,422 issued to Escamilla et al., and U.S. Pat. No. 7,311,726 issued to Mitelberg et al.

The delivery system in the above referenced Escamilla patent includes proximal, intermediate and distal cylindrical members disposed on and spaced apart along an elongated core wire such that first and second gaps are formed. The expandable stent includes anchor members which align with the gaps. The expandable stent is mounted on the intermediate cylindrical member, and the anchor members are disposed within the gaps thereby locking the stent onto the core member. In this configuration, the self-expanding stent can be pushed and pulled through the deployment catheter without damaging or deforming the stent. However, the Escamilla devices do not work well for neurovascular arteries because the elongated core is solid, the guide wire normally needed for interventional procedure can not be inserted through the device, so the elongated core wire must function as guide wire as well. It has proven difficult to design a core wire member having enough flexibility to navigate through tortuous neurovascular vessels as the guiding wire, but also enough stiffness to push the stent out of the catheter or pull the stent back into the catheter. Furthermore, the stent, in a constrained state, being mounted on the intermediate cylindrical member, may be damaged or deformed by the intermediate cylindrical member during advancement to the target disease site or during stent deployment.

Devices, such as the one shown in the above referenced Villalobos patent, use a method to reposition the stent as necessary to properly align with the target lesion to treat abdominal aortic aneurysms. The stent delivery apparatus comprises an inner shaft, outer sheath and self-expanding stent. The inner shaft has a distal end and a proximal end, the inner shaft distal end further includes at least two grooves; the self-expanding stent has a distal end and a proximal end, the self-expanding stent further includes at least two spaced apart longitudinal legs, the legs extend proximally away from the self-expanding stent, each leg including a flange. The flanges are set within the inner shaft grooves. The flange-groove combination allows the physician to partially deploy the stent while the flanges remain within the sheath, and allows physicians to pull the stent back into the delivery device if the placement is not optimal.

While the Villalobos device is designed for large vessels such as abdominal aortic, since the outer shaft size is in the range from 18 F to 24 F (6 mm-8 mm). However, neurovascular arteries are generally very tortuous and quite small, having a diameter ranging from 2.0 mm to 4.0 mm in the Circle of Willis, 2.5 to 5.5 mm in the cavernous segment of the internal carotid artery, 1.5 to 3.0 mm in the vessels of the distal anterior circulation, and 2.0 to 4.0 mm in the posterior circulation, the stent is placed at the target site via a small-diameter catheter having a lumen inner diameter of 0.7 mm or less and out diameter of 3 F (1 mm) or less. For such small size scale, it is not easy to fabricate the devices, neither assemble the devices together. Moreover, cutting such grooves into the inner shaft of a small neurovascular delivery apparatus will result in a delivery apparatus that is too weak for delivery through a tortuous pathway and that will be easily torn under the required stresses for delivery and retractable deployment of the stent. Devices disclosed in U.S. Pat. No. 6,267,783 and U.S. Pat. No. 7,311,726 have the similar disadvantages.

Accordingly, there has been a desire to have a mechanism for preventing a stent being sheared off the inner shaft while navigating to a neurovascular target site. There has been a desire to have an improved mechanism to allow a neurovascular stent to be easily pushed out of and retrieved into the small diameter delivery apparatus if the placement is not optimal. There has been a desire to have a better device to prevent the stent from being damaged or deformed during advancement through a tortuous vascular path. The following described invention provides such an improved device.

### SUMMARY OF THE INVENTION

The current invention overcomes the deficiencies of the prior art to provide a delivery apparatus enabling a self-expanding stent that is retractable during stent placement in the target neurovascular site. A conventional guide wire is preferably used in conjunction with the invention, thus the apparatus is configured to accept a guidewire. The apparatus includes an outer catheter, an inner shaft located coaxially within the outer catheter and a self-expanding stent, mounted onto the inner shaft and preloaded in its contracted state within the outer catheter's distal region. In one embodiment, the outer catheter has an outer diameter that is from about 0.037 inch to about 0.039 inch. In one embodiment, the inner shaft has an outer diameter of about 0.022 inch to about 0.0245 inch for the middle and distal sections and an outer diameter of about 0.02 inch to about 0.0205 inch for the distal portion. In a preferred embodiment, the inner shaft has an inner diameter to allow passage of the guide wire, the inner diameter being of a sufficient size for allowing the passage of a 0.014 inch or smaller diameter guidewire. In a most preferred embodiment, the inner diameter of the inner shaft is about 0.017 inch. The inner shaft includes at least one stent blocking member disposed in the distal section. The stent is mountable on the distal section of the inner shaft and preloaded within the outer catheter distal region. The self-expanding stent has proximal, middle and distal ends and is comprised of a plurality of closed cells. The closed cells may be in contact with the teeth of a gear-shaped blocking member disposed on the inner shaft so that the teeth of the gear protrude through the open space portion of a cell and can come in contact with the material portion, generally referred to as a strut, of a cell. The self-expanding stent may include locking members which interlock with a gap region formed by two or more blocking member(s) disposed on the inner shaft so as to lock the stent onto the inner shaft within the outer catheter. The stent and inner shaft are within the outer catheter and are arranged one to the other so as to enable the stent to be retractably moved out of and retrieved back to the outer catheter during placement at a target site. The invention may be used in the treatment of blood vessel blockage and aneurysms which occur in the brain. A combination of these configurations may be used, as well. Preferably, the stent locking members are radiopaque and made of radiopaque materials, such as a metal, a metal alloy, a radiopaque polymer, gold, silver, platinum, an alloy containing gold, an alloy containing silver, an alloy containing platinum or an alloy containing one or more of gold, silver and platinum. Types of radiopaque materials are known in the art, as are their uses with stent delivery. The thickness of the stent locking member shall be slightly greater than stent strut thickness. Stent cells open area are preferably larger at the ends than in the middle, in order for stent to be better interlocked with a gear shaped blocking member on the inner shaft. To treat wide neck aneurysm, preferably, the stent is covered with graft material in the middle, but not on the ends. Graft in the middle of the stent will efficiently cover aneurysm wide neck, while the stent end without being graft covered will be interlocked with the blocking members on the inner shaft. Stent graft material is made of materials such as e-PTFE, Polyurethane, etc, as is known in the art.

The material composition and construction of the outer catheter changes over the length of it to create three distinct stiffness regions: proximal, middle, and distal. The inner shaft has also three distinct sections along the length of it. The variation of the stiffness from proximal end to distal end is to achieve better trackability inside the tortuous neurovascular path. As mentioned, the inner shaft includes at least one blocking member, to prevent any relative movement between the inner shaft and stent during advancement to the target neurovascular site, and also to allow retractable delivery of the stent to the target site, wherein physicians “pull” the stent back into the delivery apparatus if the placement is not optimal. Preferably, the inner shaft has reduced diameter inner member at the distal region, so as to be extending through the contracted stent interior, to enable stent and the inner shaft to be as an integral part during movement within the outer catheter.

In an embodiment of the inner shaft, the distal section further comprises at least one blocking member, and preferably the at least one blocking member is fixedly attached to the distal section of the inner shaft. The fixedly attached blocking member can be formed as a disc or as a gear with a plurality of teeth; the number of gear teeth preferably ranging from 2 to 8. The gear teeth interlock with stent cells by fitting within the open spaces formed by the struts. The number of gear teeth will be varied to accommodate different stent cell designs. For instance, gear with 2, 3 or 6 teeth could be used for six crown design stent, gear with 2 or 4 teeth could be used for eight crown design stent. Thus, the gear shaped blocking member will be configured to interlock with a compressed self-expanding stent that is to be loaded on the inner shaft for delivery at a target site. Alternatively, the at least one blocking member is formed as a disc at the distal end of the inner shaft, and an additional blocking member is formed by varying the outer diameter of the distal section of the inner shaft.

Thus, in an embodiment, there is provided a neurovascular stent delivery apparatus, wherein said delivery apparatus comprises the inner shaft of the current invention and further comprises an outer catheter and a pre-loaded self-expanding stent. Preferably, the self-expanding stent is preloaded into the distal section of the outer catheter and is configured on the distal section of said inner shaft such that the stent is interlocked with the blocking member(s) portion of the inner shaft. In one aspect, the inner shaft comprises at least one blocking member. In one aspect, the inner shaft comprises a disc-shaped blocking member. In one aspect, the inner shaft comprises a gear shaped blocking member. In one aspect, the inner shaft comprises at least two blocking members configured along said inner shaft to form a gap section. In one aspect, a gear-shaped blocking member is positioned such that the teeth of said gear-shaped blocking member stick through the open space of the stent cells. In one aspect, a locking member on the self-expanding stent is positioned between two blocking members on the inner shaft configured to form a gap that accommodates the locking member.

One embodiment of the invention provides a neurovascular stent delivery apparatus comprising an inner shaft having a proximal portion, a middle portion and a distal portion, wherein a first blocking member is fixedly attached on the distal section, and the second blocking member is formed by varying the outer diameter of the middle and distal sections of the inner shaft so that the middle section is larger that the distal section, said first and second blocking members being configured to form a gap that interlocks with a self expanding stent comprising at least one locking member when said self-expanding stent is compressed for loading into said neurovascular stent delivery apparatus.

In one aspect of the neurovascular stent delivery apparatus, the first blocking member is disc-shaped. In one aspect of the neurovascular stent delivery apparatus, the first blocking member is gear-shaped, comprising a plurality of teeth to interlock with the cells of said compressed self expanding stent. In an aspect, the plurality of teeth is at least 2 teeth. In an aspect, the plurality of teeth is selected from the group consisting of 2 teeth, 3 teeth, 4 teeth, 5 teeth, 6 teeth, 7 teeth and 8 teeth.

In one aspect of the neurovascular stent delivery device, the first blocking member is made of a radiopaque material. In an aspect, the radiopaque material is a metal, a metal alloy, or a radiopaque polymer. In an aspect, the radiopaque material is gold, silver, platinum or an alloy containing one or more of any of the aforementioned. In one aspect of the neurovascular stent delivery apparatus, the locking member is a radiopaque material.

In one aspect of the neurovascular stent delivery apparatus, the self-expanding stent is covered with a graft material.

In one aspect of the neurovascular stent delivery apparatus wherein the first blocking member is a gear shape, the cells of the self-expanding stent are larger at the proximal and distal ends of the self-expanding stent compared to the middle section cells.

In one aspect of the neurovascular stent delivery apparatus, the inner shaft is made of a material of varying hardnesses, wherein its distal end is softest.

Another embodiment of the invention provides a neurovascular stent delivery apparatus comprising an inner shaft having a proximal end, a middle end and a distal end, wherein said middle end is configured with a gear-shaped blocking member comprising 2 or more teeth to interlock with the cells of a compressed self-expanding stent, when said stent is loaded into said neurovascular stent delivery apparatus. In one aspect of the neurovascular stent delivery apparatus, the gear shaped blocking member has 2 teeth, 3 teeth, 4 teeth, 5 teeth, 6 teeth, 7 teeth or 8 teeth.

In one aspect of the neurovascular stent delivery apparatus the gear shaped blocking member is made of a radiopaque material. In an aspect, the radiopaque material is selected from the group consisting of: a metal, a metal alloy, a radiopaque polymer, gold, silver, platinum and an alloy containing one or more of gold, silver or platinum.

In one aspect of the neurovascular stent delivery apparatus, the self expanding stent comprises a radiopaque material. In one aspect of the neurovascular stent delivery apparatus, the self expanding stent is covered with a graft material. In one aspect of the neurovascular stent delivery apparatus, the cells of the self-expanding stent are larger at the proximal and distal ends of said self-expanding stent compared to the middle section cells.

The neurovascular stent delivery apparatus described above can be used in a method for retractably delivering a self-expanding stent to a neurovascular target site, the method comprising the steps of: a.) obtaining a neurovascular delivery apparatus containing a preloaded and compressed self expanding stent, wherein said preloaded and compressed self expanding stent is interlocked with an inner shaft member of said neurovascular delivery apparatus; b.) navigating a neurovascular path towards a neurovascular target site; c.) partially deploying the preloaded and compressed self-expanding stent by pulling the outer catheter to partially expose the stent out of the distal end of an outer catheter member of a neurovascular delivery device; d.) optionally, pulling the preloaded and compressed self expanding stent back inside of said outer catheter distal end; and e.) fully deploying the preloaded and compressed self-expanding stent to the target site by pulling the outer catheter to expose the stent out of the distal end of an outer catheter member of a neurovascular delivery device.

In one aspect of the method, the neurovascular path includes one of more of a neurovascular artery, the Circle of Willis, the cavernous section of the internal carotid artery, the vessels of the distal anterior circulation and the vessels of the posterior circulation.

In one aspect of the method, the neurovascular delivery apparatus comprises an inner shaft member having a first blocking member and a second blocking member configured along said inner shaft member to form a gap section that accepts the locking member of said preloaded and compressed self expanding stent. In an aspect, the stent is partially deployed from the outer catheter distal end by applying a force to the outer catheter that causes the second blocking member to contact the locking member of the stent. In an aspect, the partially deployed stent is retracted back inside of the outer catheter by applying a force to the inner shaft that causes the first blocking member to contact the locking member of the stent and translocate the stent in a direction that is coaxal to the outer catheter lumen.

In one aspect of the method, the stent is fully deployed from the outer catheter distal end by applying a force to the outer catheter that causes the second blocking member to contact the locking member of the stent until the stent is completely outside of the outer catheter, wherein the stent expands thereby removing the locking member from the proximity of the blocking members.

In one aspect of the method, one or more of said first and second blocking members and said locking member is made from a radiopaque material.

In one aspect of the method, the neurovascular delivery apparatus comprises an inner shaft member having a first blocking member configured as a gear with a plurality of teeth that interlocks with the cells of said preloaded and compressed self expanding stent. In an aspect, the plurality of teeth is at least 2. In an aspect, the plurality of teeth is selected from the group consisting of 2, 3, 4, 5, 6, 7 and 8.

In one aspect of the method, the stent is partially deployed from the outer catheter distal end by applying a force to the outer catheter that causes the plurality of teeth to contact the strut portion of the cell. In one aspect of the method, the partially deployed stent is retracted back inside of the outer catheter by applying a force to the inner shaft that causes the plurality of teeth to contact the strut portion of the cell and translocate the stent in a direction that is coaxal to the outer catheter lumen. In one aspect of the method, the stent is fully deployed from the outer catheter distal end by applying a force to the outer catheter that causes the plurality of teeth to contact the strut portion of the cell until the stent is completely outside of the outer catheter, wherein the stent expands thereby removing the cells from the proximity of the gear teeth.

In one aspect of the method, the cells of the self-expanding stent are larger at the proximal and distal ends of the self-expanding stent compared to the middle section cells. In one aspect of the method, one or more of the first blocking member and a portion of the stent is made from a radiopaque material. In one aspect of the method, the stent is covered with a graft material.

In one aspect of the method, either or both of the outer catheter and the inner shaft comprise a plurality of sections of differing hardnesses; the distal end being the softest. In one aspect of the method, the inner shaft has a smaller outer diameter at its distal end than the outer diameter of the remainder of the inner shaft.

One embodiment of the invention provides the neurovascular stent delivery apparatus described above for use in therapy. Another embodiment of the invention provides the neurovascular stent delivery apparatus described above for use for retractably delivering a self-expanding stent to a neurovascular target site.

As will be appreciated by one skilled in the art, the method described above is equally applicable to the use of the neurovascular stent delivery apparatus. Further, certain aspects of the method will also be applicable to the delivery apparatus.

### BRIEF DESCRIPTION OF THE DRAWINGS

The foregoing and other aspects of the present invention will best be appreciated with reference to the detailed description of the invention in conjunction with the accompanying drawings, wherein:

FIG. 1 is a side view of a preferred embodiment of the delivery apparatus and a stent for retractable deployment at a target neurovascular site (not shown in FIG. 1).

FIG. 2 is a side view of another preferred embodiment of the delivery apparatus and a stent for retractable deployment at a target neurovascular site (not shown in FIG. 2).

FIG. 3 is a simplified elevational view of one preferred embodiment of the first and second blocking members disposed on the distal inner shaft, wherein the first blocking member is a disc shape.

FIG. 4 is a simplified elevational view of another preferred embodiment of the first blocking member disposed on the inner shaft, wherein the first blocking member is a gear shape.

FIG. 5 is a simplified elevational view of another preferred embodiment of the first blocking member disposed on the inner shaft, wherein the first blocking member is a gear shape.

FIG. 6 is a simplified elevational view of another preferred embodiment of the first blocking member disposed on the inner shaft, wherein the first blocking member is a gear-shape.

FIG. 7 is partial cross-sectional views showing the deployment of the closed-cell self-expanding stent with stent marker placement.

FIG. 8 is partial cross-sectional views of another preferred embodiment showing the deployment of the closed-cell self-expanding stent with stent marker placement.

FIG. 9 is side view of inner shaft.

FIG. 10 is side view of outer catheter.

### DETAILED DESCRIPTION OF THE INVENTION

As is used herein, the terms “about” or “approximate” when used to describe the dimensions of the described device mean that the size of the device need not be precisely the dimensions described. Those of skill in the art will understand from this disclosure how to design embodiments of the invention with varied dimensions. Such is within the spirit of this current invention.

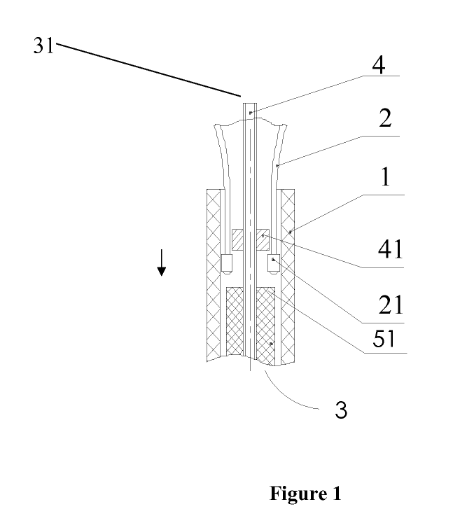

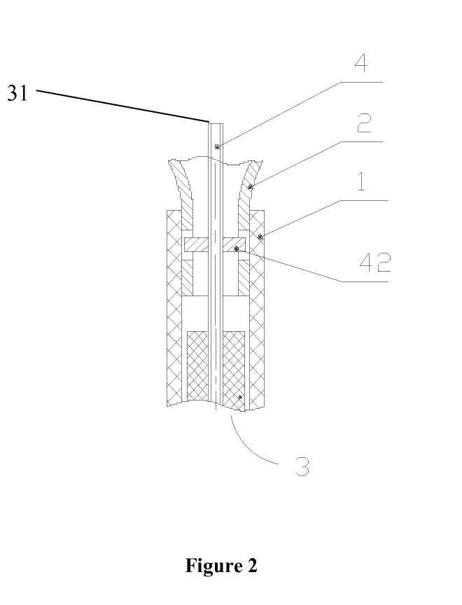

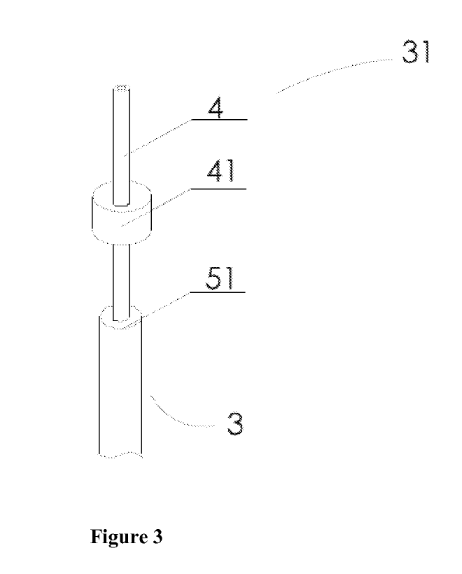

FIG. 1 and FIG. 2 illustrate two exemplary embodiments of a neurovascular self expanding stent delivery apparatus of the current invention. In FIG. 1 there is seen a distal section 4 and a middle section 3 of an inner shaft 31. Attached to the inner shaft 31 there is a first blocking member 41. FIG. 3 is partial exploded views of the distal end of the inner shaft 31, wherein the blocking member 41 is a disc-shape disposed on the inner shaft. Returning to FIG. 1, the larger outer diameter of the middle section 3 of inner shaft 31 compared to the outer diameter of the distal section 4 provides a second blocking member 51. First blocking member 41 and second blocking member 51 are configured along the inner shaft 31 so that the first and second blocking members act in conjunction with one another to form a gap that accepts locking member 21 of a stent 2. In use, interlocking the stent 2 with the inner shaft 31 by placing the locking member 21 into the gap formed by the first and second blocking members 41, 51 allows for retractable release of said stent 2 from the outer catheter 1 to a neurovascular target site.

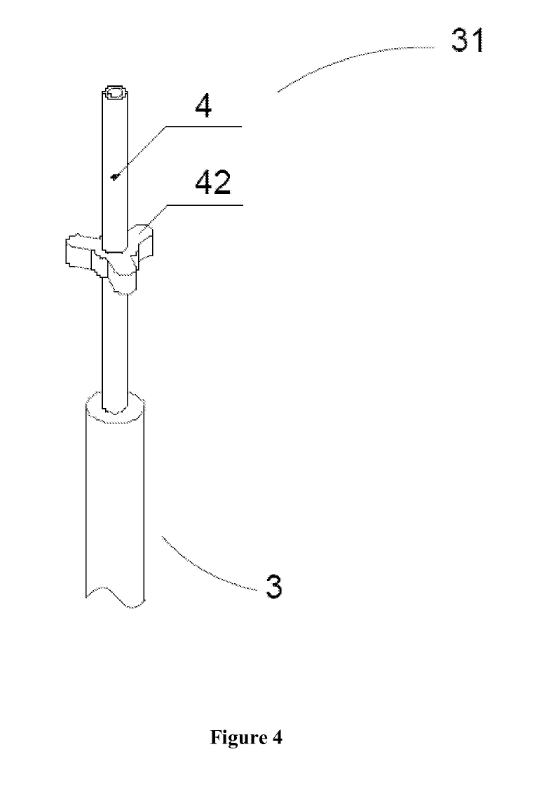

FIG. 2 illustrates another preferred embodiment of self expanding stent delivery apparatus of the current invention. In FIG. 2, there is seen a distal section and a middle section of an inner shaft 31. Attached to the inner shaft 31 there is a first blocking member 42. In this embodiment, the first blocking member 42 is shown protruding through the stent 2. In this way, the blocking member 42 interlocks with the stent 2 and allows for retractable release of said stent 2 from the outer catheter 1. Exemplary first blocking members 42 that can be used with this embodiment include a gear-shaped member, some of which are shown in FIGS. 4-6. When the first blocking member 42 is a gear-shaped member, the gear-shape can be configured to have at least 2 teeth, more preferably from 2 teeth to 8 teeth. The number, the size, the shape and the placement of the teeth for the gear-shaped first blocking member 42 are preferably configured to fit within the open space of the cells of a stent. FIGS. 4, 5 and 6 are partial exploded views of the distal end of the inner shaft. The blocking members 42 disposed on the inner shaft takes the gear form in three, four and six teeth configurations respectively.

For use with an inner shaft of the current invention, wherein said inner shaft is configured with at least one blocking member to interlock the inner shaft with the stent, there is described a stent. Preferably, the stent is a neurovascular stent; more preferably a collapsible, self-expanding neurovascular stent; more preferably still a collapsible, self-expanding, closed-cell neurovascular stent. Such stents and methods for constructing such stents are known in the art. Preferably, the stent is configured to be pre-loaded on the inner shaft of the current invention and within the distal portion of the outer catheter so as to be retractable when used with a apparatus comprising an inner shaft of the current invention. The purpose of radio-opaque markers is to allow a user to identify the location of a marked object when said object is in vivo. Thus, a radiopaque marker on outer catheter 1 will allow for visualization of the catheter relative a target delivery location. Further, a radiopaque marker on the stent 2 will allow for visualization of the stent relative a target location. When the inner shaft 31, the stent 2 and the outer catheter 1 are all marked with a radiopaque marker, the relative positioning of these devices one to the other is more easily determined.

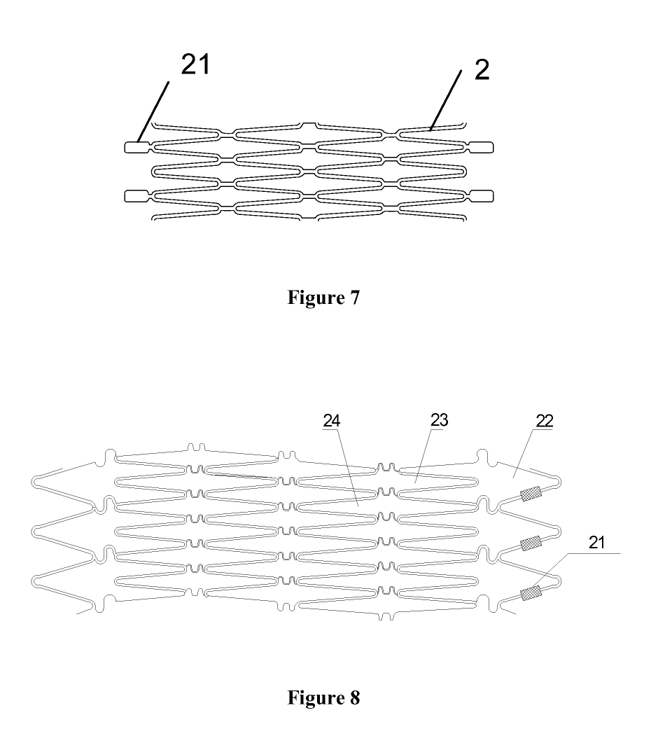

FIG. 7 illustrates a self expanding stent 2 comprising locking members 21 at the most proximal end. Locking members 21 are preferably placed on the proximal end of a self-expanding stent 2. More preferably, stent 2 will comprise at least two locking members 21. Most preferably, stent 2 will comprise a plurality of proximal legs, each of said proximal legs comprising a locking member 21. Preferably, said self-expanding stent is preloaded on the inner shaft 31 and within the distal end of an outer catheter 1. The length of inner shaft is preferably about 2-50 cm longer than outer catheter 1. Turning back to FIG. 1, there is a self-expanding stent 2 comprising a locking member 21 as illustrated in FIG. 7, and the stent is showed interlocked with the inner shaft 31. In FIG. 1 stent 2 is shown in releasable contact with inner shaft 31 and partially delivered from within the distal section of outer catheter 1 wherein locking member 21 is positioned between blocking members 41 and 51. In such an arrangement, the stent 2 can be partially delivered out of catheter 1 by pulling the outer catheter 1 while the inner shaft 31 remains stationary. This movement applies a force between blocking members 51 and locking member 21. So long as the stent 2 has not been fully released out of catheter 1, then stent 2 can be retracted back into catheter 1 by keeping the outer catheter 1 stationary while the inner shaft 31 is pulled, thereby applying force between blocking members 41 and locking member 21 to retract the stent 2. Confinement of locking member 21 within the inner diameter of catheter 1 provides a force to cause stent 2 to remain in a collapsed position, particularly so at its proximal end wherein the locking member 21 resides. This confinement provides for locking member 21 to remain positioned in the gap between blocking members 41 and 51. Once stent 2 is fully delivered from within the inner diameter of catheter 1, the stent will expand, thus removing locking member 21 from its position within the gap of blocking members 41 and 51.

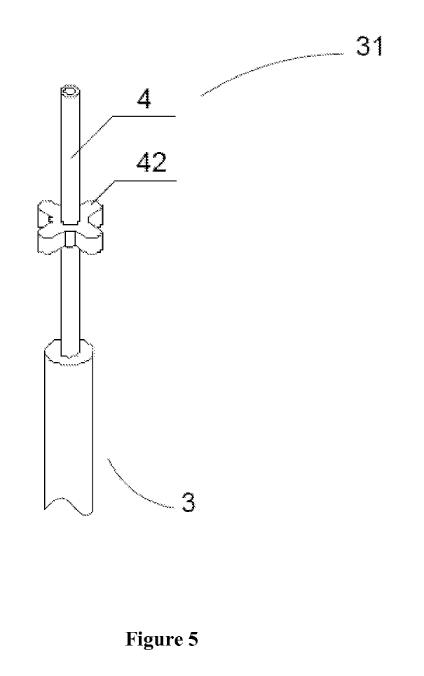

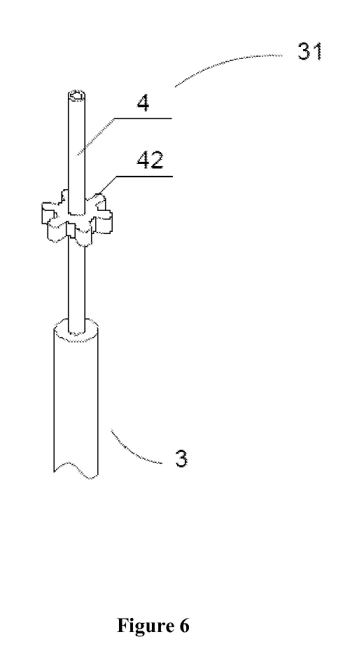

FIG. 8 illustrates a self expanding stent comprising locking members near the proximal end. At least one stent marker 21 is attached to the proximal stent strut. The radiopaque marker 21 thickness will be slightly larger than that of stent strut. The stent cell 22 at the proximal end is larger than the stent cells 23, 24 in the middle, so as for the blocking member 42 fixed in the inner shaft 31 to be easily interlocked together with. As illustrated in FIG. 4, first blocking member 42 has 3 gear teeth; as illustrated in FIG. 5, first blocking member 42 has 4 gear teeth; as illustrated in FIG. 6, first blocking member 42 has 6 gear teeth. First blocking member 42 will preferably have 2-8 gear teeth. For instance, a gear with 2, 3 or 6 teeth could be used for six crown design stents, and a gear with 2 or 4 teeth could be used for eight crown design stents. The outside diameter of the first blocking member 42 shall be smaller than then inner diameter of the outer catheter 1. First blocking member 42 teeth could be fabricated in different geometry shapes. Turning back to FIG. 2, there is a self-expanding stent 2 as illustrated in FIG. 8. The first blocking member 42 gear teeth are interlocked with stent 2 in the area of stent cell 22. In such an arrangement, the stent 2 can be released out of catheter 1 by pulling the outer catheter 1 while the inner shaft 31 remains stationary. So long as the stent 2 has not been fully released out of catheter 1, then stent 2 can be retracted back into catheter 1. When the physician wants to “pull” the partial expanded stent back into the outer catheter 1, inner shaft 31 is pulled proximately, and the gear teeth of the first blocking member 42 will engage stent 2 in the area of stent cell 22 by coming into contact with the strut members of the cell, and force the stent 2 to retreat back into the outer catheter 1. As a result, stent 2 will be recaptured within the outer catheter 1. To treat wide neck aneurysm, preferably, stent is covered with graft material in the middle, but not on the ends (not shown in FIG. 8). Graft in the middle of the stent will efficiently cover aneurysm wide neck, while the stent end without being graft covered will be interlocked with the blocking members on the inner shaft. Stent graft material is made of materials such as e-PTFE, Polyurethane, etc. The first blocking member 42 fixed on the distal section 4 of the inner shaft 31 will interlock with cells at a more proximal end of stent 2 wherein the stent is not covered by graft material, such that graft material will not be damaged by first blocking member 42.

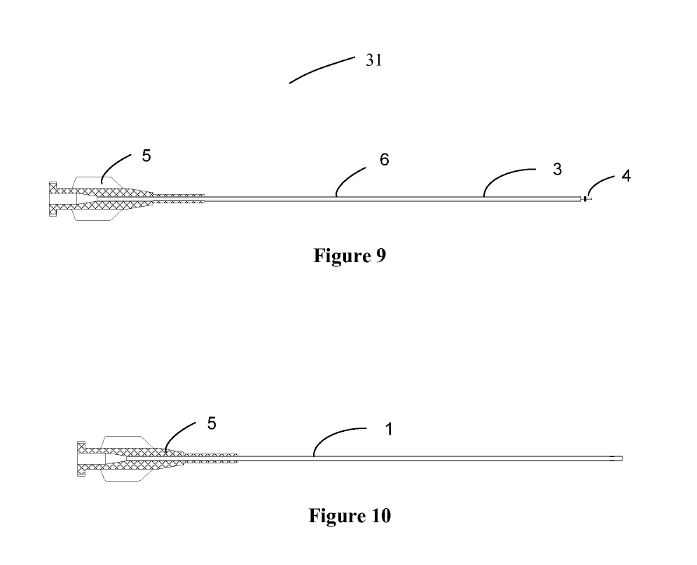

As is better seen in FIG. 9, the inner shaft is designed for navigating tortuous paths, such as a tortuous neurovascular path, to deliver a self-expanding stent. The inner shaft 31 is designed to have a plurality of sections wherein said sections provide both strength and flexibility so as to deliver a stent through a tortuous neurovascular path and without damaging the stent. In order to achieve this objective, inner shaft 31 is generally constructed as follows. This proximal section 6 is preferably a metal hypotube material made of materials such as nintinol, stainless steel. The inner diameter of inner shaft 31 should be a sufficient dimension to allow smooth passage of a guidewire. The inner diameter may further comprise a lubricious inner liner to aid in smooth passage of the guidewire Inner shaft 31 is further constructed to comprise a middle section 3 that provides exceptional flexibility, trackability and kink resistance. This middle section is preferably made of materials such as polyether block amide material, and, optionally, is a polyether block amide material over a braided metal wire reinforcement Inner shaft 31 is further constructed to comprise a distal section 4 that is of a length within the range of sizes from about 3 cm to about 5 cm. This distal section is flexible and strong material, preferably a polyimide material, and has an outer diameter of about 0.0165 inch and an inner diameter that is sufficiently sized to allow smooth passage of a 0.014 guidewire. Inner shaft 31 also preferably comprises a radio-opaque marker, which is preferably, but not necessarily, located on first blocking member 41 or 42.

As is better seen in FIG. 10, the outer shaft 1 is designed for navigating tortuous paths, such as a tortuous neurovascular path, to deliver a self-expanding stent. The outer shaft 1 is bonded to hub 5. In one preferred embodiment, the outer catheter 1 is configured to provide a plurality of sections. The proximal section is preferably a rigid stiffness polyether block amide, such as Pebax® 7233, and covers a braided wire reinforcement. The inner diameter should be a sufficient dimension to allow for smooth passage of an inner shaft 31 as described herein. More preferably, the inner diameter should be of a sufficient dimension to allow for smooth passage of an inner shaft 31 as described herein and further comprising a preloaded compressed closed cell neurovascular stent for delivery to a target location. The inner diameter may further comprise a lubricious inner liner to aid in smooth passage of the inner shaft and/or preloaded stent. In one example, such an inner liner is constructed from a PTFE material having at least a 0.001 inch wall thickness. Outer catheter 1 further comprises a middle section. The middle section is preferably a medium stiffness polyether block amide, such as Pebax® 5533, and covers a braided wire reinforcement. The inner diameter of this middle section may further comprise a PTFE liner having a wall thickness of about 0.001 inch.

Turning to FIG. 10, there is shown an outer catheter 1 designed for navigating tortuous pathways to deliver a self-expanding stent. In the figure outer catheter 1 is bonded to hub 5, Outer catheter 1 provides a proximal section, middle section and a distal section. In an exemplary embodiment, the proximal section is about 93 cm in length and comprises a rigid stiffness polyether amide such as Pebax 7233 covering a braided wire reinforcement. The braided wire reinforcement is preferably about 0.001 inch in height, about 0.003 inch in length and has a braid density of about 60-100±5 ppi. The proximal section has an outer diameter of about 0.039 inch and an inner diameter of about 0.029 inch, thereby allowing for smooth passage through a tortuous path and retractable deployment of an inner shaft comprising a preloaded self expanding closed cell neurovascular stent. The inner portion of the outer catheter 1 preferably comprises a lubricious inner liner to aid in smooth passage of the inner shaft and preloaded stent. One exemplary lubricious liner is PTFE material having a wall thickness of about 0.001 inch. In an exemplary embodiment, the middle section is about 23 cm in length, has an outer diameter of about 0.037 inch, has an inner diameter of about 0.029 inch, and comprises a medium stiffness polyether block amide such as Pebax 5533 covering a braided wire. Preferably the braided wire is 0.001 by 0.003 inch with a 60-100±5 ppi braid density. Preferably, the inner portion of the outer catheter middle section comprises a lubricious liner such as PTFE with a wall thickness of about 0.001 inch. In an exemplary embodiment, outer catheter 1 comprises a distal section that is about 20 cm in length, has an outer diameter of about 0.037 inch, and has an inner diameter of about 0.029 inch. The distal section is preferably a soft stiffness polyether block amide, such as Pebax® 2533 covering a braided wire reinforcement, as described. The inner portion of this distal section may further comprise a PTFE liner having a wall thickness of about 0.001 inch. Outer catheter 1 also preferably comprises at least one radio-opaque marker, preferably located at the distal end of said outer catheter 1.

In one exemplary embodiment, the invention provides a neurovascular stent delivery apparatus, wherein said delivers apparatus includes an outer catheter, and an inner shaft located coaxially within the outer catheter; a stent is mounted on the distal section of the inner shaft and preloaded within the outer catheter distal region, wherein the inner shaft includes at least one stent blocking member disposed in the distal section, wherein the self-expanding stent has proximal and distal end and is comprised of a plurality of closed cells and further includes locking members, wherein the blocking members fixed on the inner shaft form a gap that accepts the locking member of the self-expanding stent and allows for retractable release of said stent back into the outer catheter, wherein optionally, the blocking member on the inner shaft is in gear form with various number of teeth, such that the blocking member is embedded within the said stent cell in a configuration that allows the blocking member fixed on the inner shaft to engage the stent when the inner shaft is pulled proximately, and wherein one or both of the stent and the first blocking member comprises a radio-opaque marker.

In one exemplary embodiment there is a neurovascular stent delivery apparatus, wherein said delivery apparatus includes an outer catheter and an inner shaft located coaxially within the outer catheter; a compressed self-expanding stent comprising a plurality of closed cells mounted on the distal section of the inner shaft and preloaded within the outer catheter distal region; wherein the inner shaft comprises one or more blocking members disposed in the distal section. In one aspect, the self-expanding stent comprises at least one locking member. In one aspect, the at least one locking member is made of a radiopaque material.

In one aspect wherein the inner shaft comprises two or more blocking members, the first blocking member having a disc-shape and being located on the inner shaft at a more distal position relative to the second blocking member such that the first and second blocking members are configured to provide a gap between the first and second blocking members, the gap being sufficient to receive a locking member portion of the compressed self-expanding stent. In one aspect a first of the one or more blocking members is a gear-shape comprising a plurality of teeth configured to interlock with the cells of said compressed self-expanding stent.

In one aspect, the self-expanding stent is covered with a graft material. In one aspect, at least one of the one or more blocking members is made of a radiopaque material. In one aspect, said inner shaft is made of material of varying hardness, and said distal section is the softest. In one aspect, the outer catheter is made of material of varying hardness, wherein said distal section is the softest. In one aspect, the cells of said self-expanding stent are larger at the proximal ends compared to the cells of the middle section of the stent. In one aspect, the outer catheter covers a braided wire or coil reinforcement. In one aspect, the stent has at least one radiopaque marker at the proximal ends.

In one aspect, the delivery apparatus includes an outer catheter, and an inner shaft located coaxially within the outer catheter; a stent mounted on the distal section of the inner shaft and preloaded within the outer catheter distal region, wherein the inner shaft includes at least one stent blocking member disposed in the distal section, wherein the self-expanding stent has proximal and distal ends and is comprised of a plurality of closed cells and further includes locking members, wherein the blocking members fixed on the inner shaft form a gap that accepts the locking member of the self-expanding stent and allows for retractable release of said stent back into the outer catheter, wherein optionally, the blocking member on the inner shaft is in gear form with various number of teeth, such that the blocking member is embedded within the said stent cell in a configuration that allows the blocking member fixed on the inner shaft to engage stent when inner shaft is pulled proximately, and wherein one or both of the stent or the first blocking member comprises a radio-opaque marker.

In one embodiment, any neurovascular stent delivery apparatus described herein for use in therapy. In one embodiment, any neurovascular stent delivery apparatus described herein for use for retractably delivering a self-expanding stent to a neurovascular target site.

What is claimed is:

1. A neurovascular stent delivery apparatus, wherein said delivers apparatus includes an outer catheter, and an inner shaft located coaxially within the outer catheter; a stent is mounted on the distal section of the inner shaft and preloaded within the outer catheter distal region, wherein the inner shaft includes at least one stent blocking member disposed in the distal section, wherein the self-expanding stent has proximal and distal end and is comprised of a plurality of closed cells and further includes locking members, wherein the blocking members fixed on the inner shaft form a gap that accepts the locking member of the self-expanding stent and allows for retractable release of said stent back into the outer catheter, wherein optionally, the blocking member on the inner shaft is in gear form with various number of teeth, such that the blocking member is embedded within the said stent cell in a configuration that allows the blocking member fixed on the inner shaft to engage stent when inner shaft is pulled proximately, and wherein one or both of the stent or the first blocking member comprises a radio-opaque marker.

2. The neurovascular stent delivery apparatus of claim 1, wherein the self-expanding stent is covered with graft material at a portion of the stent selected from the group consisting of: the proximal portion and distal portion, and the proximal portion, middle portion and distal portion.

3. The neurovascular stent delivery apparatus of claim 2, wherein the graft material is made of material selected from the group consisting of: e-PTFE and Polyurethane.

4. The neurovascular stent delivery apparatus of claim 1, wherein the blocking member is gear-shaped and has plurality of teeth ranging from 2 to 8 and configured to accommodate a stent cell design.

5. The neurovascular stent delivery apparatus of claim 1, wherein the blocking member is made of materials such as metal, metal alloy, or radiopaque polymers.

6. The neurovascular stent delivery apparatus of claim 1, wherein the stent cells are larger at the ends of the stent than in the middle.

7. The neurovascular stent delivery apparatus of claim 1, wherein the outer shaft is designed with a material of differing stiffness from proximal end to distal end, distal end being the softest, and wherein the differing stiffness of the outer catheter is configured for navigating tortuous paths, such as a tortuous neurovascular path, to deliver a self-expanding stent

8. The neurovascular stent delivery apparatus of claim 1, wherein one or more of the radiopaque markers are made of gold, silver, platinum or its alloy.

9. The neurovascular stent delivery apparatus of claim 1, wherein the stent has at least one radiopaque marker at the proximal ends.

10. The neurovascular stent delivery apparatus of claim 1, wherein the blocking members are radiopaque.

11. The neurovascular stent delivery apparatus of claim 1, wherein the outer catheter covers a braided wire or coil reinforcement.

12. The neurovascular stent delivery apparatus of claim 1, wherein the blocking members are made of metal, metal alloy, or radiopaque polymers.

13. A neurovascular stent delivery apparatus, wherein said delivers apparatus includes an outer catheter and an inner shaft located coaxially within the outer catheter; a compressed self-expanding stent comprising a plurality of closed cells is mounted on the distal section of the inner shaft and preloaded within the outer catheter distal region; wherein the inner shaft comprises one or more blocking members disposed in the distal section.

14. The delivery apparatus of claim 13, wherein at least one of the one or more blocking members is made of materials such as metal, metal alloy, or radiopaque polymers.

15. The delivery apparatus of claim 13, wherein the self-expanding stent of comprises at least one locking member.

16. The delivery apparatus of claim 15, wherein the locking members are made of radiopaque materials.

17. The delivery apparatus of claim 13, wherein a first of the one or more blocking members is a disc-shape and is located on the inner shaft at a more distal position relative to a second blocking member, thereby configured to provide a gap between the first and second blocking members, the gap being sufficient to receives a locking member portion of the compressed self expanding stent.

18. The delivery apparatus of claim 13, wherein a first of the one or more blocking members of is a gear-shape comprising a plurality of teeth configured to interlock with the cells of said compressed self-expanding stent.

19. The delivery apparatus of claim 13, wherein the self-expanding stent is covered with a graft material.

20. The delivery apparatus of claim 13, wherein said inner shaft is made of material of varying hardness, wherein said distal section is softest.

21. The delivery apparatus of claim 13, wherein said out catheter is made of material of varying hardness, wherein said distal section is softest.

22. The delivery apparatus of claim 13, wherein said self-expanding stent cells are larger at the proximal ends compared to the middle section cells.

23. A method for retractably delivering a self-expanding stent to a neurovascular target site, comprising the steps of:
a. obtaining a neurovascular delivery apparatus containing a preloaded and compressed self expanding stent, wherein said preloaded and compressed self expanding stent is interlocked with an inner shaft member of said neurovascular delivery apparatus;
b. navigating a neurovascular path towards a neurovascular target site;
c. partially delivering said preloaded and compressed self-expanding stent by pulling the outer catheter to partially expose the stent out of the distal end of an outer catheter member of a neurovascular delivery device;
d. optionally, retracting said partially released stent back inside of said outer catheter distal end; and
e. fully delivering said preloaded and compressed self-expanding stent to said target site by pulling the outer catheter to expose the stent out of the distal end of an outer catheter member of a neurovascular delivery device.

24. The method of claim 23, wherein said neurovascular delivery apparatus comprises an inner shaft having a first blocking member and a second blocking member configured along said inner shaft to form a gap section that accepts the locking member of said preloaded and compressed self expanding stent.

25. The method of claim 24, wherein said stent is partially deployed from said outer catheter distal end by pulling the outer catheter proximately, while the inner shaft is held in a fixed position.

26. The method of claim 25, wherein said partially deployed stent is pulled back inside of said outer catheter by applying a force to the inner shaft that cause the first blocking member to contact the locking member of said stent and retract the said stent back into said outer catheter.

27. The method of claim 23, wherein said neurovascular delivery apparatus comprises an inner shaft having first blocking member configured as a gear with a plurality of teeth that interlocks with the cells of said preloaded and compressed self expanding stent.

28. The method of claim 27, wherein said plurality of teeth is at least 2.

29. The method of claim 28, wherein said plurality of teeth is selected from the group consisting of 2, 3, 4, 5, 6, 7 and 8.

30. The method of claim 27, wherein said stent is partially deployed from said outer catheter distal end by pulling the outer catheter proximately, while the inner shaft is held in a fixed position.

31. The method of claim 30, wherein said partially deployed stent is pulled back inside of said outer catheter by applying a force to the inner shaft that causes said plurality of teeth to contact the strut portion of the cell and retract the said stent back into said outer catheter.

32. The method of claim 27, wherein said cells are larger at the proximal end of said self-expanding stent compared to the middle section cells.
